(19) United States
(12) Patent Application Publication
(10) Pub. No.: US 2016/0154015 A1
(43) Pub. Date: Jun. 2, 2016

(54) MICROFLUIDIC DEVICE TO DETECT CANNABIS IN BODY FLUIDS

(71) Applicants: Kathleen Stitzlein, Millersburg, OH (US); Tri Tien Quang, Akron, OH (US)

(72) Inventors: Kathleen Stitzlein, Millersburg, OH (US); Tri Tien Quang, Akron, OH (US)

(21) Appl. No.: 14/956,847

(22) Filed: Dec. 2, 2015

Related U.S. Application Data
(60) Provisional application No. 62/086,341, filed on Dec. 2, 2014.

(51) Int. Cl. G01N 33/94; B01L 3/00

(52) U.S. Cl. CPC G01N 33/948; B01L 3/502715; B01L 2200/10; B01L 2300/0636

(57) ABSTRACT

A microfluidic device for on-site quantitative detection of marijuana in body fluids has a body fluid collecting reservoir for receiving a body fluid and an anti-THC antibody reservoir that retains anti-THC antibodies. A first micromixer receives the body fluid from the body fluid collecting reservoir and the anti-THC antibodies from the anti-THC antibody reservoir to form a sample mixture. The device further comprises a fluorescein-THC conjugate reservoir that fluorescein-THC conjugate; a second micromixer that receives the sample mixture and the fluorescein-THC conjugate to form a test mixture; and at least one light source to emit an excitation wavelength and at least one detector to receive an emission wavelength.

### CROSS-REFERENCE TO RELATED APPLICATIONS

This application claims the benefit of U.S. Provisional Application No. 62/086,341 filed on Dec. 2, 2014, the content of which is incorporated herein by reference.

### FIELD OF THE INVENTION

The present invention generally relates to a novel device for on-site detection of tetrahydrocannabinol (THC) in body fluids. Specifically, the present invention relates to a microfluidic device for the on-site detection of THC in saliva. In some embodiments, the present invention utilizes a fluorescein-THC conjugate to quantitatively indicate the concentration of THC in a sample based on a competitive immunoassay.

### BACKGROUND OF THE INVENTION

Tetrahydrocannabinol (THC), as shown in FIG. 1a, is the main active chemical found in marijuana. As marijuana use becomes legal in more states across the country and across the world, the need for a quick, accurate, and sensitive roadside method to detect levels of THC in drivers will continue to increase. THC and its metabolite THC-COOH remain detectable in the saliva of a marijuana user for approximately 12-24 hours after use and it has been found that the amount of THC in saliva corresponds to the length of time the drug remains active in the body.

Marijuana is the most frequently consumed illegal drug. Therefore, THC is an indispensable target for most drug testing systems. Although urine tests for marijuana were established years ago, there is a growing need for new rapid tests administered as easily as a breath alcohol test. Such tests must be able to detect low concentrations of free THC in saliva (approximately 4-50 ng/mL) rather than detecting water-soluble THC metabolites, which occur in urine many hours after marijuana ingestion or inhalation. However, the detectability of THC is severely limited by its poor solubility in water (only 2.8 μg/mL).

As a result of its poor solubility in water, THC molecules are adsorbed on proteins, cell debris and other impurities of biological samples or they are attached to surfaces, e.g. of sample collection tools or vials. Compared to rapid tests for other drugs of abuse, the limit of detection (LOD) for THC is limited. Therefore, a fast and sensitive detection of THC in biological samples like saliva is a challenge for most drug testing systems.

The standard laboratory method for detection of THC is gas chromatography/liquid chromatography mass spectrometry (GC/LC-MS). However, for on-the-spot detection immediately after sample collection, some other methods should be applied. At present, heterogeneous lateral flow immunoassays are the most used and commercially successful formats for rapid drug testing. These assays are easy to use, they deliver quick results, and in most cases they obviate the need to use a complicated apparatus for incubation and detection (e.g. pumps, valves, CCD, etc.). However, most lateral flow assays are only qualitative or semi-quantitative. Heterogeneous immunoassay formats like enzyme-linked immunosorbent assay (ELISA) achieve a large dynamic measurement range and quantitative data, but they need many additional steps like immobilization, washing, and the addition of several reagents to complete one test. ELISA is a common laboratory technique which is used to measure the concentration of an analyte (usually antibodies or antigens) in solution.

Therefore, there is a need in the art for a quick, accurate, quantitative, and sensitive method to detect levels of THC in bodily fluids.

### SUMMARY OF THE INVENTION

In a first embodiment, the present invention provides a microfluidic device for on-site quantitative detection of marijuana in body fluids comprising: a body fluid collecting reservoir for receiving a body fluid; a anti-THC antibody reservoir retaining anti-THC antibodies; a first micromixer receiving said body fluid from said body fluid collecting reservoir and receiving said anti-THC antibodies from said anti-THC antibody reservoir to form a sample mixture; a fluorescein-THC conjugate reservoir retaining fluorescein-THC conjugate; a second micromixer receiving said sample mixture and said fluorescein-THC conjugate to form a test mixture; and at least one light source to emit an excitation wavelength and at least one detector to receive an emission wavelength.

In a second embodiment, the present invention provides a microfluidic device as in the first embodiment, further comprising a first channel providing fluid communication between the body fluid collecting reservoir and the first micromixer; a second channel providing fluid communication between the anti-THC antibody reservoir and the first micromixer; and a third channel providing fluid communication between F-THC reservoir and the second micromixer.

In a third embodiment, the present invention provides a microfluidic device as in either the first or second embodiment, further comprising at least one pump.

In a fourth embodiment, the present invention provides a microfluidic device as in any of the first through third embodiments, wherein the at least one pump is a dual peristaltic micropump.

In a fifth embodiment, the present invention provides a microfluidic device as in any of the first through fourth embodiments, wherein the dual peristaltic micropump assists in moving the body fluid and the anti-THC antibodies from the respective first channel and second channel to the first micromixer.

In a sixth embodiment, the present invention provides a microfluidic device as in any of the first through fifth embodiments, wherein further comprising a second pump wherein the second pump assists in moving the fluorescein-THC conjugate to the second micromixer.

In a seventh embodiment, the present invention provides a microfluidic device as in any of the first through sixth embodiments, wherein the second pump is a single peristaltic micropump.

In an eighth embodiment, the present invention provides a microfluidic device as in any of the first through seventh embodiments, further comprising a dual sensor system along the first channel and second channel to ensure that at least a minimum amount of the body fluid and the anti-THC antibodies respectively are traveling into the first micromixer.

In a ninth embodiment, the present invention provides a microfluidic device as in any of the first through eighth embodiments, wherein the minimum amount of the body fluid and the anti-THC antibodies is from about 0.5 microliters (μL) or more to about 1.0 μL or less.

In a tenth embodiment, the present invention provides a microfluidic device as in any of the first through ninth embodiments, further comprising a single sensor system along the third channel to ensure that at least a minimum amount of the fluorescein-THC conjugate is traveling into the second micromixer.

In an eleventh embodiment, the present invention provides a microfluidic device as in any of the first through tenth embodiments, wherein the minimum amount of the fluorescein-THC conjugate is about from about 0.5 microliters (μL) or more to about 1.0 μL or less.

In a twelfth embodiment, the present invention provides a microfluidic device as in any of the first through eleventh embodiments, further comprising a first reaction chamber adjacent the first micromixer to hold the sample mixture for a set amount of time to allow for a complete reaction of the body fluid and the anti-THC antibodies.

In a thirteenth embodiment, the present invention provides a microfluidic device as in any of the first through twelfth embodiments, wherein the set amount of time is between about 1 minute and about 4 minutes

In a fourteenth embodiment, the present invention provides a microfluidic device as in any of the first through thirteenth embodiments, further comprising a second reaction chamber adjacent the second micromixer to hold the test mixture for a set amount of time to allow for a complete reaction of the sample mixture and the fluorescein-THC conjugate.

In a fifteenth embodiment, the present invention provides a microfluidic device as in any of the first through fourteenth embodiments, wherein the set amount of time is between about 1 minute and about 4 minutes.

In a sixteenth embodiment, the present invention provides a microfluidic device as in any of the first through fifteenth embodiments, wherein the at least one light source and at least one detector are adjacent to a second side of the second micromixer to detect the presence of marijuana, or they lack thereof, in the test mixture.

In a seventeenth embodiment, the present invention provides a microfluidic device as in any of the first through sixteenth embodiments, wherein the first light source is a light emitting diode (LED).

In an eighteenth embodiment, the present invention provides a microfluidic device as in any of the first through seventeenth embodiments, wherein a second light source and a second detector are adjacent to a first side of the second micromixer to detect the quenched fluorescence intensity of the fluorescein-THC conjugate and the sample mixture.

In a nineteenth embodiment, the present invention provides a microfluidic device as in any of the first through eighteenth embodiments, wherein the second light source is a light emitting diode (LED).

In a twentieth embodiment, the present invention provides a microfluidic device as in any of the first through nineteenth embodiments, wherein further comprising a waste reservoir to collect the test mixture once the microfluidic device has finished detecting the presence, or lack thereof, of marijuana in the body fluid.

In a twenty-first embodiment, the present invention provides a microfluidic device as in any of the first through twentieth embodiments, further comprising a control means, wherein the control means comprises a main circuit board, a start/stop button, a clean button to clean the device, and a display screen.

### BRIEF DESCRIPTION OF THE DRAWINGS

FIG. 1a provides the chemical structure of tetrahydrocannabinol;

FIG. 1b provides the chemical structure of a fluorescein-THC conjugate;

FIG. 2 provides a fluid schematic view of an embodiment of the microfluidic device of the present invention; and

FIG. 3 provides an electrical schematic view of an embodiment of the microfluidic device of the present invention.

### DETAILED DESCRIPTION OF ILLUSTRATIVE EMBODIMENTS

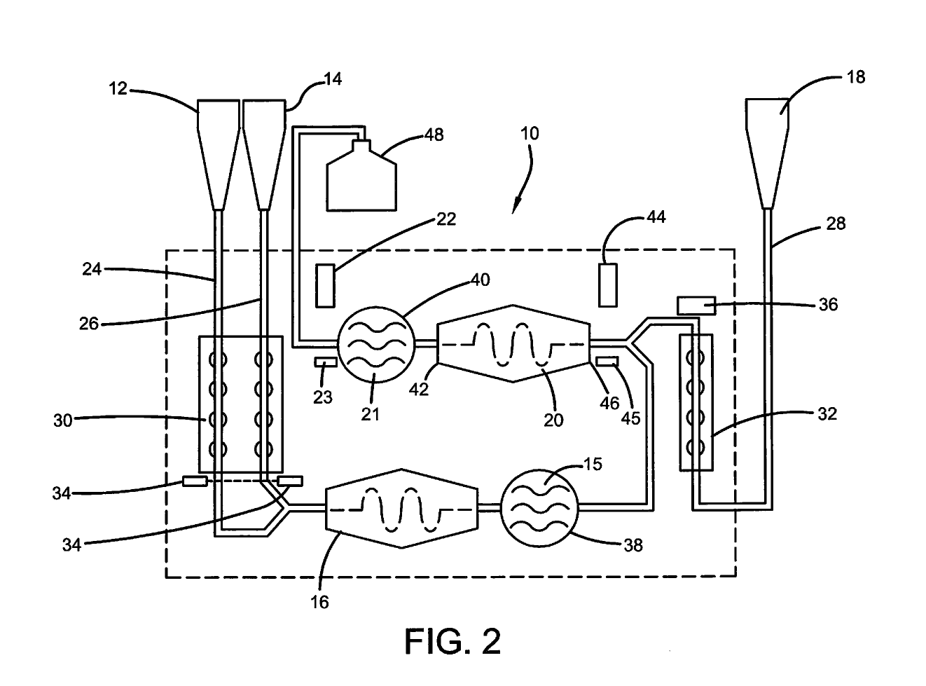

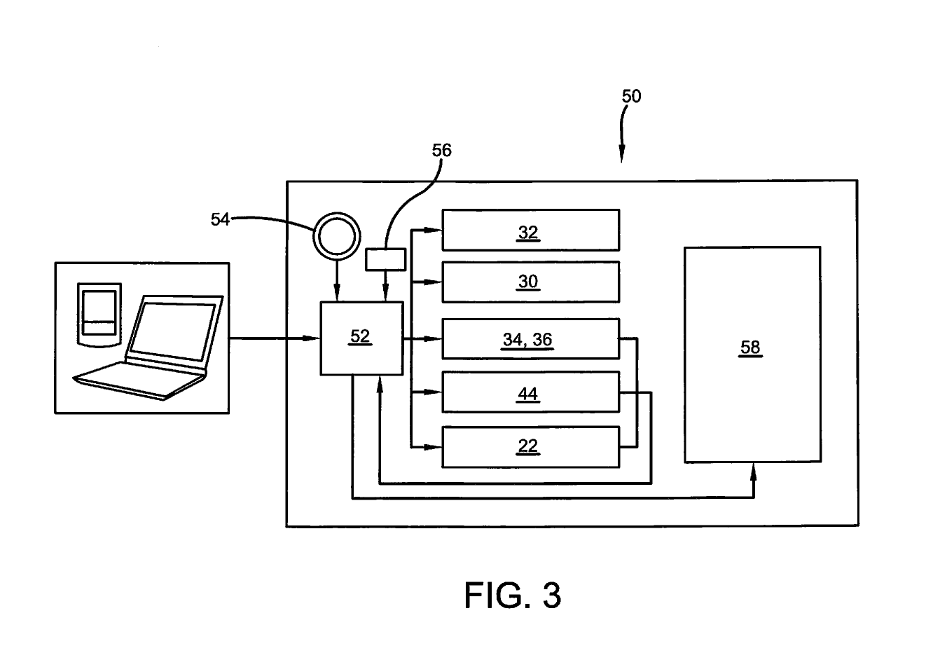

FIG. 2 provides a schematic drawing of one embodiment of the microfluidic device 10 of the present invention. The device 10 comprises a body fluid collecting reservoir 12 for receiving a body fluid and an anti-THC antibody reservoir 14 for retaining anti-THC antibodies. In one embodiment the body fluid that is received in the body fluid collecting reservoir 12 is saliva and, in some embodiments, the reservoir is designed so that the subject to be tested can deposit their saliva directly from their mouth into the body fluid collecting reservoir 12. In some embodiments, the body fluid reservoir 12 can retain from 50 microliters (μL) or more to 100 μL or less of body fluid. In some embodiments, the anti-THC antibody reservoir 14 can retain from 1 milliliters (ml) or more to 2 ml or less of anti-THC antibodies. The anti-THC antibody reservoir 14 will hold enough anti-THC antibodies to be able to run multiple tests on the microfluidic device 10.

In some embodiments, the anti-THC antibody is any antibody that is suitable for capturing THC of F-THC molecules. In some embodiments, the anti-THC antibody is selected from the group consisting of mouse anti-THC monoclonal antibody, goat anti-THC monoclonal antibody, sheep anti-THC monoclonal antibody and mixtures thereof. In some embodiments, the anti-THC antibody is selected from mouse anti-THC monoclonal antibody.

The device 10 further comprises a first micromixer 16 which receives the body fluid, such as saliva, from the body fluid collecting reservoir 12 and the anti-THC antibodies from the anti-THC antibody reservoir 14 to form a sample mixture 15. In one embodiment, the first micromixer 16 has a serpentine coil design to induce chaotic advection for an enhancement of mixing time. If the body fluid contains THC, then the anti-THC antibodies will bind to the THC in the sample mixture 15, and, if the body fluid does not contain THC, the anti-THC antibodies will have nothing to bind to and will remain unbound in the sample mixture 15.

A fluorescein-THC conjugate reservoir 18 retains a fluorescein-THC conjugate. In some embodiments, the fluorescein-THC conjugate reservoir 18 retains from 1 ml or more to 2 ml or less of fluorescein-THC conjugate. The fluorescein-THC conjugate reservoir 18 will hold enough fluorescein-THC conjugate to be able to run multiple tests on the microfluidic device 10.

In some embodiments, the fluorescein-THC conjugate is any fluorescein that shows low fluorescence response in aqueous solutions, but releases the fluorescence if the THC residue of F-THC is bound by an anti-THC antibody. In some embodiments, the fluorescein-THC conjugate is any fluorescein that has optimal fluorescence intensity with separate emission and excitation wavelengths, which reduces any possible background fluorescence interference, and which will conjugate easily with 11-Nor-cannabinol. The fluorescein chosen should reduce any possible background fluorescence interference. In some embodiments, the fluorescein-THC is 11-Nor-cannabinol-9-carboxylic derivatives.

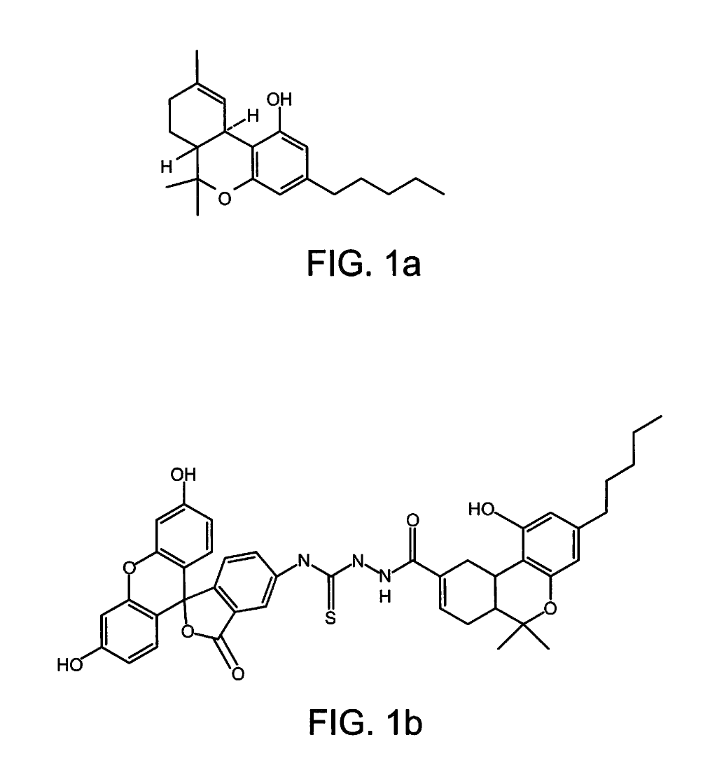

FIG. 1B provides an 11-Nor-cannabinol-9-carboxylic derivative (herein abbreviated as F-THC). The F-THC shows only a weak fluorescence emission in aqueous solutions due to fluorescein fluorescence quenching by intramolecular THC. However, the fluorescence can be released if the THC residue of the F-THC is bound by an anti-THC antibody. Depending on the reaction conditions, the ratio of released and quenched fluorescence intensity varies between 2.5 and 5. This increase of fluorescence can be used as an indicator of the formation of F-THC-antibody complex.

The anti-THC antibody selectively binds with both free THC and F-THC. Thus, when F-THC is introduced to a drug free sample mixture 15 the antibody reacts with F-THC and strong fluorescence is observed. For drug positive sample mixtures 15, the formation of the F-THC-antibody complex is inhibited due to the binding of free THC and anti-THC antibody, which results in a weaker fluorescence. The fluorescence intensity is 2.5-5 times greater when the F-THC-antibody complex is formed as compared to the quenched fluorescence intensity observed when free THC is present in the sample mixture 15.

The change of fluorescence intensity of F-THC due to the interaction of F-THC and anti-THC antibodies suggest that the quenching effect is related with intramolecular interactions. Fluorescence quenching induced by dimer formation of fluorescence dye conjugate fusillade-coumarin only occurs at concentrations over 1 μM. F-THC is also in the quenched state at concentrations in nanomolar range or lower; at these concentrations, formations of a dimer is not probable.

To introduce fluorescein-THC conjugate, the device 10 further comprises a second micromixer 20 which receives the sample mixture 15, formed in the first micromixer 16, and the fluorescein-THC conjugate from the fluorescein-THC conjugate reservoir 18 to form a test mixture 21. In one embodiment, the second micromixer 20 has a serpentine coil design to induce chaotic advection for an enhancement of mixing time. The fluorescein-THC conjugate will bind with free anti-THC antibodies that are in the sample mixture 15. If the sample mixture 15 does not contain any THC, then the fluorescein-THC conjugate will bind to the ant-THC antibodies, and there will be a strong fluorescence detected; however, if the sample mixture 15 does contain THC, the formation of the fluorescein-THC conjugate-antibody complex will be inhibited due to the binding of the THC and the anti-THC antibody, and a weaker florescence will be detected.

In some embodiments, florescence is detected by using at least one combination light source 22 and detector 23. The term “combination” is employed because each light source 22 would have an associated detector 23. Each light source 22 employed provides an excitation wavelength causing a fluorescence response in the test mixture 21. This fluorescence response is detected by the detector 23 of the combination light source 22 and detector 23 in order to detect the strength of the fluorescence response. The amount of THC in the test mixture 21 is not actually being “determined” per se, but a change in fluorescence is being observed by the combination light source 22 and detector 23 thus relating to either the complete lack of THC in the test mixture 21 (evidenced by a strong fluorescence) or the presence of THC in the test mixture 21 (evidenced by a inhibited fluorescence). This can provide notice of THC in the test mixture 21 and thus in the test subject.

To seek quantitative information regarding THC levels, experimental test samples, with known amounts of THC in the test sample, can be prepared to perform measurements with the device 10 to find the difference of fluorescence intensities corresponding to the known concentrations of THC in the experimental test samples. Correlation curves can then be prepared based on the collected data. Based on the correlation curves, a formula to accurately determine the concentration of THC in a test sample can be prepared.

In some embodiments, the detector 23 has a lens and an optical filter. In some embodiments, the detector 23 is further connected to a microprocessor. The fluorescence response emitted from test mixture 21 is captured by the lens, which aids in focusing the emitted signal onto the detector 23. In some embodiments, the lens is a PCX, a camera, or lens system of the like.

In some embodiments, an optical filter is placed between the lens and the detector 23, wherein the field of view is projected by the lens onto the detector 23 through the optical filter. The optical filter is provided to filter out the excitation spectra from the emission spectra expected from the F-THC employed. Thus, the emission spectrum, as perceived by the detector 23, is not negatively impacted by the excitation spectra. The filtering reduces the amount of fluorescence signal onto the detector 23. The use of a long-pass filter on the detector 23 will increase the fluorescence signal, enabling better detection of lower concentrations.

In some embodiments, the optical filter is an emission filter. Emission filters allow for the transmission of the emitted fluorescence from the test mixture 21 into the detector 23, while rejecting the excitation spectra from the light source 22. The selection of the excitation filter is such that the filter allows for the transmission of the excitation spectra and rejects wavelengths in the emission spectra.

In some embodiments, the optical filter is an optical bandpass filter. In other embodiments, the optical filter may be selected from the group consisting of any filter which allows for the transmission of excitation spectra and rejects wavelengths of the emission spectra.

In some embodiments, the optical filter is a filter wheel placed between the lens and the detector 23. A filter wheel allows for easy removal and installation of multiple filters within the detector 23 to obtain the appropriate filter for filtering out the excitation spectra from the emission spectra expected from the fluorescence dye employed. In some embodiments, a user can switch between filters on the filter wheel manually, or in other embodiments, operation of the filter wheel is automated.

In some embodiments, the detector 23 detects the fluorescence response emitted from the test mixture 21. After passing through an optical filter, the fluorescent response is projected onto the detector 23. The total optical power incident onto the detector 23 depends on the total fluorescence signal emitted from the test mixture 21. The projection of the lens onto the detector 23 is then used to obtain the total fluorescence signal incident onto the detector 23. The detector 23 produces a signal to be compared to the standard curve to compare for quantification. The amount of THC in the test mixture 21 is not actually being “determined” per se, but a change in fluorescence is being observed by the detector 23, thus relating to either the complete lack of THC in the test mixture 21 (evidenced by a strong fluorescence) or the presence of THC in the test mixture 21 (evidenced by a inhibited fluorescence).

In some embodiments, the detector 23 is a silicon photodiode. In other embodiments, the detector 23 may be selected from the group consisting of UV-VIS, biolumen, fiber optic cable, APD (avalanche photodiode), PMT (photomultiplier tube), cell-phone camera, tablet PC, or the like.

In some embodiments, the light source 22 includes an optical filter. The optical filter is placed in front of the light source 22 to limit the spectra of the illumination to the excitation spectra of the fluorescent dye, such that only wavelengths within the excitation spectra are transmitted.

In some embodiments, the light source 22 includes a light source driver. The power spectrum of the light source 22 determines the shape and intensity of the fluorescence emission signal. The fluorescence response of the test mixture 21 depends upon the wavelength and optical power of the incident light emitted from the light source 22. Broadband sources such as, halogen lamps, xenon arc lamps and mercury arc lamps can be used to obtain spectra of the fluorescence emission signals. The intensity of the fluorescence response at wavelength of maximum emission is used for THC estimation.

In some embodiments, the light source 22 is a monochromatic light source. The fluorescence response from the test mixture 21 depends upon the wavelength and optical power of the incident light from the light source 22. In some embodiments, the light source 22 includes an adjustable power source to induce fluorescence. In some embodiments, the light source 22 is a light emitting diode (LED), in other embodiments, a laser, a fluorescent lamp or broadband source of the like.

In preferred embodiments, the light source 22 is an LED. Two main parameters, brightness and wavelength of maximum emission, determine the choice of LED to be used in the microfluidic device 10. LEDs provide high output power at the wavelength of maximum emission. In some embodiments, the LED can be selected from a super bright yellow LED or any LED selected based on the excitation spectra of the dye.

In some embodiments, the light source 22 includes at least one LED, in other embodiments, two or more LEDs, and still in other embodiments, multiple LEDs may be chosen to provide uniform illumination. By increasing the number of LEDs present in the light source 22, the current supply (or the inflow of electrons) increases, thereby increasing the number of photons emitted from the test mixture 21, which will produce a stronger fluorescence response.

In LED applications, it may be found that a stable current supply is required for providing power to the light source 22, as the performance of light source 22 depends upon the stability of the current supplied to it. In some embodiments, a light source driver supplies a linear current to the light source 22 providing temperature stability, i.e. the current remains stable over a temperature range thereby maintaining the brightness of the light source 22 over that temperature range. The light source driver further maintains a stable current supplied to the light source 22, thereby producing a constant output.

In some embodiments, the light source driver is adjustable. By increasing the current supply (or the inflow of electrons) into the light source 22, the number of photons emitted from the test mixture 21 will increase, thereby increasing the strength of the fluorescence response.

In some embodiments, the light source 22 is an LED light source and includes a light source driver to maintain a stable current. A stable current supply is required for providing power to the LED light source, as the performance of the LED light source depends upon the stability of the current supplied to it. The light source driver supplies a linear current to the LED light source providing temperature stability, i.e. the current remains stable over a temperature range thereby maintaining the brightness of the LED light source over that temperature range. The light source driver further maintains a stable current supplied to the LED light source, thereby producing a constant output.

In some embodiments, the microfluidic device 10 further comprises a first channel 24, a second channel 26, and a third channel 28. The first channel 24 provides fluid communication between the body fluid collecting reservoir 12 and the first micromixer 16; the second channel 26 provides fluid communication between the anti-THC antibody reservoir 14 and the first micromixer 16; and the third channel 28 provides fluid communication between the F-THC reservoir 18 and the second micromixer 20.

In some embodiments, the microfluidic device 10 further comprises at least one pump 30 to assist in moving the body fluid from the first channel 24 and the anti-THC antibodies from the second channel 26 to the first micromixer 16. In specific embodiments, the at least one pump 30 is a dual peristaltic micropump.

In some embodiments, the microfluidic device 10 further comprises a second pump 32 to assist in moving the fluorescein-THC conjugate to the second micromixer 20. In specific embodiments, the second pump 32 is a single peristaltic micropump.

In some embodiments, the microfluidic device 10 further comprises a dual sensor system 34 along the first channel 24 and second channel 26 to ensure that at least a minimum amount of the body fluid and the anti-THC antibodies respectively are traveling into the first micromixer 16. In specific embodiments, if the dual sensor system 34 detects that a minimum amount of the body fluid and the anti-THC antibodies respectively are not traveling into the first micromixer 16, then the dual sensor system 34 will alert the user of the microfluidic device 10 that not enough body fluid and/or anti-THC antibodies are traveling into the first micromixer 16. Such an alert will then allow the user to add more body fluid and/or anti-THC antibodies into the microfluidic device 10. In some embodiments, the minimum amount of the body fluid and the anti-THC antibodies needed to travel into the first micromixer 16 is about 0.5 microliters (μL) or more to about 1.0 μL or less.

In some embodiments, the microfluidic device 10 further comprises a single sensor system 36 along the third channel 28 to ensure that at least a minimum amount of the fluorescein-THC conjugate is traveling into the second micromixer 20. In specific embodiments, if the single sensor system 36 detects that a minimum amount of the fluorescein-THC conjugate is not traveling into the second micromixer 20, and then the single sensor system 36 will alert the user of the microfluidic device 10 that not enough fluorescein-THC conjugate is traveling into the second micromixer 20. Such an alert will then allow the user to add more fluorescein-THC conjugate into the microfluidic device 10. In some embodiments, the minimum amount of the fluorescein-THC needed to travel into the second micromixer 20 is about 0.5 microliters (μL) or more to about 1.0 μL or less.

In some embodiments, the microfluidic device 10 has a first reaction chamber 38 adjacent the first micromixer 16 to hold the sample mixture 15 for a set amount of time so as to allow for a complete reaction of the body fluid and the anti-THC antibodies of the sample mixture 15. In some embodiments, the set amount of time that the sample mixture will be held in the first reaction chamber 38 is between about 1 minute and about 4 minutes. This reaction chamber may or may not be employed depending upon residence time and mixing of the sample mixture achieved by the length of the fluid paths to and through the first micromixer 16. The sample mixture 15 is shown in the first reaction chamber 38 in FIG. 2 because that is the most practical place to show the sample mixture 15 in FIG. 2, regardless of the fact that the first reaction chamber 38 may not be present.

In some embodiments, the microfluidic device 10 has a second reaction chamber 40 adjacent the second micromixer 20 to hold the test mixture 21 for a set amount of time so as to allow for a complete reaction of the sample mixture and the fluorescein-THC conjugate. In some embodiments, the set amount of time that the test mixture 21 will be held in the second reaction chamber 40 is between about 1 minutes and about 4 minutes. This second reaction chamber may or may not be employed depending upon residence time and mixing of the test mixture 21 achieved by the length of the fluid paths to and through the second micromixer 20. The test mixture 21 is shown in the second reaction chamber 40 in FIG. 2 because that is the most practical place to show the text mixture 21 in FIG. 2, regardless of the fact that the second reaction chamber 40 may not be present.

In some embodiments, the light source 22 and detector 23 the microfluidic device 10 is adjacent to a second side 42 of the second micromixer 20 to detect the presence of marijuana, or the lack thereof, in the test mixture 21. In yet another embodiment, the microfluidic device 10 includes a second light source 44 and a second detector 45 adjacent to a first side 46 of the second micromixer 20 to detect the presence of any background interference in the sample mixture 15 and fluorescein-THC conjugate. The second light source 44 and the second detector 45 measures quenched fluorescence and the light source 22 and detector 23 measures released fluorescence.

In some embodiments, the microfluidic device 10 further contains a waste reservoir 48 to collect the test mixture 21 once the microfluidic device 10 has finished detecting the presence, or lack thereof, of marijuana in a body fluid.

It will be appreciated that the control of all elements of the device 10 can be implemented in known ways, typically with some or all controls being implemented through various hardware and/or software and/or firmware, all represented and designated herein as a processor 50. One or more processors can be used, and it is to be appreciated that reference to “processor 50” encompasses as well the use of any number of appropriate processors, hardware, software, and firmware. The processor 50 can control the pumps 30, 32, the light sources 22, 44, the detectors 23, 45 and the sensor systems 34, 36 to advance fluids through the device as described and analyze fluorescence responses to qualitatively determine (again according to the teaching above) the presence of THC in the sample fluid from the test subject. The processor 50 and associate hardware and/or firmware and/or software can carry out an automated process once the test subject has deposited fluid in the fluid collecting reservoir 12.

In some embodiments providing for quantitative analysis, the standard curve to determine THC concentration is programmed within the processor 50, wherein the fluorescence response is used to determine the concentration of the THC directly from the data stored in the processor 50. In short, a human need not compare fluorescence intensity to a standard curve, but rather the data relevant to the standard curve is loaded into the processor 50, which can simply provide a resultant THC concentration by calculating THC concentration based on the standard curve data.

In some embodiments, the processor 50 includes a main circuit board 52, a start/stop button 54, an optional clean button 56 to clean the device, and a display screen 58. In some embodiments, the display screen 58 is a cellular phone.

In light of the foregoing, it should be appreciated that the present invention significantly advances the art by providing a microfluidic device to quantitatively detect cannabis in body fluids that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A microfluidic device for on-site quantitative detection of marijuana in body fluids comprising:
a. a body fluid collecting reservoir for receiving a body fluid;
b. a anti-THC antibody reservoir retaining anti-THC antibodies;
c. a first micromixer receiving said body fluid from said body fluid collecting reservoir and receiving said anti-THC antibodies from said anti-THC antibody reservoir to form a sample mixture;
d. a fluorescein-THC conjugate reservoir retaining fluorescein-THC conjugate;
e. a second micromixer receiving said sample mixture and said fluorescein-THC conjugate to form a test mixture; and
f. at least one light source to emit an excitation wavelength and at least one detector to receive an emission wavelength.

2. The microfluidic device of claim 1, further comprising a first channel providing fluid communication between the body fluid collecting reservoir and the first micromixer; a second channel providing fluid communication between the anti-THC antibody reservoir and the first micromixer; and a third channel providing fluid communication between F-THC reservoir and the second micromixer.

3. The microfluidic device of claim 2, further comprising at least one pump.

4. The microfluidic device of claim 3, wherein the at least one pump is a dual peristaltic micropump.

5. The microfluidic device of claim 4, wherein the dual peristaltic micropump assists in moving the body fluid and the anti-THC antibodies from the respective first channel and second channel to the first micromixer.

6. The microfluidic device of claim 5, further comprising a second pump wherein the second pump assists in moving the fluorescein-THC conjugate to the second micromixer.

7. The microfluidic device of claim 3, wherein the second pump is a single peristaltic micropump.

8. The microfluidic device of claim 2, further comprising a dual sensor system along the first channel and second channel to ensure that at least a minimum amount of the body fluid and the anti-THC antibodies respectively are traveling into the first micromixer.

9. The microfluidic device of claim 8, wherein the minimum amount of the body fluid and the anti-THC antibodies is from about 0.5 microliters (μL) or more to about 1.0 μL or less.

10. The microfluidic device of claim 2, further comprising a single sensor system along the third channel to ensure that at least a minimum amount of the fluorescein-THC conjugate is traveling into the second micromixer.

11. The microfluidic device of claim 10, wherein the minimum amount of the fluorescein-THC conjugate is about from about 0.5 microliters (μL) or more to about 1.0 μL or less.

12. The microfluidic device of claim 1, further comprising a first reaction chamber adjacent the first micromixer to hold the sample mixture for a set amount of time to allow for a complete reaction of the body fluid and the anti-THC antibodies.

13. The microfluidic device of claim 12, wherein the set amount of time is between about 1 minute and about 4 minutes.

14. The microfluidic device of claim 1, further comprising a second reaction chamber adjacent the second micromixer to hold the test mixture for a set amount of time to allow for a complete reaction of the sample mixture and the fluorescein-THC conjugate.

15. The microfluidic device of claim 14, wherein the set amount of time is between about 1 minute and about 4 minutes.

16. The microfluidic device of claim 1, wherein the at least one light source and at least one detector are adjacent to a second side of the second micromixer to detect the presence of marijuana, or they lack thereof, in the test mixture.

17. The microfluidic device of claim 16, wherein the first light source is a light emitting diode (LED).

18. The microfluidic device of claim 14, wherein a second light source and a second detector are adjacent to a first side of the second micromixer to detect the quenched fluorescence intensity of the fluorescein-THC conjugate and the sample mixture.

19. The microfluidic device of claim 18, wherein the second light source is a light emitting diode (LED).

20. The microfluidic device of claim 1, further comprising a waste reservoir to collect the test mixture once the microfluidic device has finished detecting the presence, or lack thereof, of marijuana in the body fluid.

21. The microfluidic device of claim 1, further comprising a control means, wherein the control means comprises a main circuit board, a start/stop button, a clean button to clean the device, and a display screen.
